(19) United States
(12) Patent Application Publication
(10) Pub. No.: US 2007/0270841 A1
(43) Pub. Date: Nov. 22, 2007

(54) Implantable devices

(75) Inventor: Behnam Badie, LaCanada, CA (US)

Correspondence Address: Medlen & Carroll, LLP, Suite 350, 101 Howard Street, San Francisco, CA, 94105, US

(73) Assignee: Madison Surgical Designs, LLC, La Canada, CA (US)

(21) Appl. No.: 11/700,463

(22) Filed: Jan. 31, 2007

Related U.S. Application Data
(60) Provisional application No. 60/763,510, filed on Jan. 31, 2006.

(51) Int. Cl. A61B 17/58

(52) U.S. Cl. 606/61; 606/69

(57) ABSTRACT

The present invention relates to implantable devices, and particularly implantable devices that may be positioned, for example, into the sphenoid sinus or sella turcica of a subject. In certain embodiments, the implantable devices comprise a center plate and a plurality of protruding arms extending from the center plate, both of which may be composed of bioresorbable material. In particular embodiments, the implantable devices are configured to help reconstruct the sellar floor after it has been damaged in order to prevent cerebrospinal fluid leaks.

The present application claims priority to U.S. Provisional Application Ser. No. 60/763,510, filed Jan. 31, 2006, which is herein incorporated by reference in its entirety.

### FIELD OF THE INVENTION

The present invention relates to implantable devices, and particularly implantable devices that may be positioned, for example, into the sphenoid sinus or sella turcica of a subject. In certain embodiments, the implantable devices comprise a center plate and a plurality of protruding arms extending from the center plate, both of which may be composed of bioresorbable material. In particular embodiments, the implantable devices are configured to help reconstruct the sellar floor after it has been damaged in order to prevent cerebrospinal fluid leaks.

### BACKGROUND OF THE INVENTION

Reconstruction of the cranial base is often necessary during or after transsphenoidal surgery for many reasons, such as the preventing the occurrence of cerebrospinal fluid rhinorrhea, and to maintain anatomic integrity. For example, reconstruction of the floor of the sella turcica is often necessary following pituitary tumor removal surgery. This type of reconstruction has historically been accomplished with the aid of cartilage or bone harvested from the nasal septum or sphenoid sinus which is used to maintain the position of sealant material such as adipose tissue or muscle which may be supplemented with fibrin glue. Introduction of the endonasal endoscopic approach to cranial base type surgery, however, generally does not expose such cartilaginous or bony material for reconstruction of the sellar floor. As such, the use of synthetic implants have been introduced to aid in the reconstruction process (e.g., see, e.g., Kaptain et al., Neurosurgery, 48(1):232-234, 2001; Kubo et al., J. Neurosurg., 102:938-939, 2005; Cappabianca et al., Neurosurgery, 51:1365-1372, 2002; and Abe et al., No. Shinkei Geka, June; 29(6):511-5, 2001; all of which are herein incorporated by reference for all purposes). The use of the minimally invasive endonasal endoscopic approach in the narrow nasal conduit, however, restricts surgical manipulation. This makes it difficult to precisely handle synthetic implants that are used. Therefore, what is needed are implantable devices that are easy to manipulate in the nasal conduit and that are useful in reconstructing the sellar floor.

### SUMMARY OF THE INVENTION

The present invention provides implantable devices, and particularly implantable devices that may be positioned, for example, into the sphenoid sinus or sella turcica of a subject. In certain embodiments, the implantable devices comprise a center plate and a plurality of protruding arms extending from the center plate, both of which may be composed of bioresorbable material. In particular embodiments, the implantable devices are configured to help reconstruct the sellar floor after it has been damaged in order to prevent cerebrospinal fluid leaks.

In some embodiments, the present invention provides implant devices comprising; a) a center plate composed of a first biocompatible material (e.g. bioresorbable material) that is preferably inflexible or substantially inflexible at room temperature, wherein the center plate has: i) a length between about 4.0 and 40 millimeters, ii) a width between about 3.0 and 30 millimeters, and iii) a thickness of about 0.3 to about 5.0 millimeters, and b) a plurality of protruding arms composed of a second biocompatible material (e.g. bioresorbable material) that is inflexible at room temperature, wherein the plurality of protruding arms are attached to, or integral with, the center plate, and wherein the plurality of protruding arms project from the center plate in a planar manner. In other embodiments, the length, width, and thickness encompass larger sizes (i.e. they are not limited to the sizes recited above). In certain embodiments, the length is between about 4.0 and 14.0 millimeters. In particular embodiments, the width is between about 3.0 and 13.0 millimeters. In other embodiments, the plurality of protruding arms comprises at least 3 protruding arms (e.g. 3, 4, 5, . . . 10, etc.). In certain embodiments, the plurality of protruding arms comprises at least 4 protruding arms. In further embodiments, the plurality of protruding arms consists of 4 protruding arms. In additional embodiments, the plurality of protruding arms comprises first and second pairs of protruding arms, wherein the first pair is attached to a first side of the center plate, and the second pair is attached to a second side of the center plate such that the second pair is situated directly across the center plate from the first pair.

In certain embodiments, the first and/or second biocompatible material is selected from the group consisting of: ceramics (e.g., alumina ceramic), silicone, polyester-silicone, stainless steel, titanium, and amporphous polymers. In some embodiments, the first and/or second biocompatible material is bioresorbable material. In particular embodiments, the bioresorbable material comprises polylactide. In particular embodiments, the polylactide comprises poly D, L-lactide acid (PDLLA). In certain embodiments, the first and/or second biocompatible material comprises RESORBX (KLS Martin, L.P.). In other embodiments, the first and second biocompatible materials are the same. In additional embodiments, the first and/or second biocompatible material is malleable at a temperature of 70 degrees Celsius.

In certain embodiments, the plurality of protruding arms are integral with the center plate. In other embodiments, the center plate comprises at least one plate hole (e.g., 1, 2, 3, . . . 10 . . . 20, etc). In particular embodiments, the at least one plate hole or holes has a shape selected from: a circle, oval, square, rectangle, octagon, and similar shapes. In additional embodiments, the size of the plate hole or holes in the center plate is between about 3.0 and 12.0 millimeters squared (e.g. 3.0 . . . 5.0 . . . 8.0 . . . 12.0 millimeters squared). In particular embodiments, the plate hole or holes are configured to be grasped by a surgical device, such as surgical forceps. In further embodiments, the center plate does not have any plate holes (e.g., its a solid plate).

In some embodiments, the plurality of protruding arms are inflexibly attached to the center plate. In certain embodiments, the plurality of protruding arms are flexibly attached to the planar center plate (e.g. such that they will bend under stress).

In other embodiments, the center plate has a length between about 4.0 and about 50 millimeters (e.g., 4.0 . . . 10.0 . . . 20 . . . 30 . . . 40 . . . and 50 millimeters), a width between about 4 and about 40 millimeters (e.g., 4.0 . . . 10 . . . 20 . . . 30 . . . and 40 millimeters), and a thickness between about 0.1 and 8.0 millimeters (e.g. 0.1 . . . 0.9 . . . 1.5 . . . 2.5 . . . 4.5 . . . 7.0 . . . and 8.0 millimeters). In particular embodiments, the center plate had a thickness of about 0.6 millimeters. In certain embodiments, the center plate is planar. In other embodiments, the center plate has a length of about 10 to 12 millimeters. In further embodiments, the center plate had a width of about 5-7 millimeters. In other embodiments, at least two of the plurality of protruding arms have a length between about 2.5 and about 5 millimeters, and a width of about 0.5 to about 2.0 millimeters. In further embodiments, the protruding arms have a thickness between about 0.5 and about 2.0 millimeters. In some embodiments, the protruding arms have a thickness that is about 0.6 millimeters. In additional embodiments, the thickness of the protruding arms is about the same, or exactly the same, as the center plate. In certain embodiments, at least one of the plurality of protruding arms comprises grooves. In further embodiments, the grooves are configured to facilitate cutting away a portion of the protruding arm. In some embodiments, at least one protruding arm extends from the center plate in a plane parallel to, and above, the plane of the center plate (e.g. 0.2 millimeters above), and at least one protruding arm extends from the center plate in a plane parallel to, and below, the plane of the center plate (e.g. 0.2 millimeters below). In certain embodiments, the plurality of protruding arms are configured to secure the implant device to bone by extending above and below the bone (e.g., as shown in FIG. 3).

In some embodiments, the implant devices are configured to help reconstruct the skull base of a subject. In particular embodiments, the implant devices are configured to be placed into the spenoid sinus of a subject. In other embodiments, the implant devices are configured to be placed into the sella turcica of a subject. In certain embodiments, the implant devices are configured to be placed into the ethmoid sinus of a subject. In some embodiments, the implant devices are configured to aid in preventing cerebrospinal fluid leaks in a subject. In certain embodiments, the implant devices are configured to aid in reconstruction of the floor of the sella turcica. In further embodiments, the center plate has a shape selected from: a circle, oval, square, rectangle, octagon, and similar shapes.

In certain embodiments, the present invention provides methods comprising; a) providing; i) a subject, and ii) an implant device comprising a plurality of protruding arms, wherein the implant device is configured to be inserted into the skull base of the subject to aid in preventing cerebrospinal fluid leaks in the subject; and b) inserting the implant device into the skull base of the subject.

In some embodiments, the present invention provides methods comprising; a) providing; i) a subject, and ii) an implant device comprising; A) a center plate composed of a first biocompatible material that is inflexible at room temperature, wherein the center plate has: i) a length between about 4.0 and 40.0 millimeters, ii) a width between about 3.0 and 30.0 millimeters, and iii) a thickness of about 0.3 to about 5.0 millimeters, and B) a plurality of protruding arms composed of a second biocompatible material that is inflexible at room temperature, wherein the plurality of protruding arms are attached to, or integral with, the center plate, wherein the plurality of protruding arms project from the center plate in a planar manner; and b) inserting the implant device into the skull of the subject (e.g., the sphenoid sinus or sella turcica of the subject). In certain embodiments, the inserting is into the sinus of a subject.

In certain embodiments, the inserting is performed during an endoscopic transsphenoidal procedure. In other embodiments, the inserting is via the nose of the subject. In additional embodiments, the implant device is inserted into the skull base of the subject. In further embodiments, the implant device is inserted into the sphenoid sinus of the subject. In some embodiments, the implant device is inserted into the sella turcica of the subject. In other embodiments, the implant device is inserted into the ethmoid sinus of the subject. In particular embodiments, the sellar turcica comprises a sellar floor that is damaged, and the inserting comprises positioning the implant device into the sellar turcica in order to reconstruct the damaged sellar floor. In other embodiments, the inserting is conducted after a pituitary tumor is at least partially removed from the sellar turcica. In further embodiments, the inserting the implant device in the sphenoid sinus or sellar turcia aids in preventing cerebrospinal fluid leaks in the subject.

In some embodiments, the present invention provides systems comprising; a) a sterilized implant device comprising; i) a center plate composed of a first biocompatible material that is inflexible at room temperature, wherein the center plate has: i) a length between about 4.0 and 40.0 millimeters, ii) a width between about 3.0 and 30.0 millimeters, and iii) a thickness of about 0.3 to about 5.0 millimeters, and ii) a plurality of protruding arms composed of a second biocompatible material that is inflexible at room temperature, wherein the plurality of protruding arms are attached to, or integral with, the center plate, wherein the plurality of protruding arms project from the center plate in a planar manner; and b) a first sealed container, wherein the sterilized implant device is located inside the first sealed container, wherein the first sterilized implant device remains sterile while located inside the first sealed container. In certain embodiments, the system further comprises c) a temperature indicator, wherein the temperature indicator visually indicates if the first sealed container has been exposed to a threshold temperature at any point. In further embodiments, the temperature indicator is a WARMMARK temperature indicator from IntroTech (Loenen, Netherlands). In other embodiments, the systems further comprise a second sealed container, wherein the first sealed container is located inside of the second sealed container, and wherein the temperature indicator is affixed to the second sealed container.

In some embodiments, the present invention provides methods for making an implant device comprising; a) providing; i) an implant device pattern, ii) biocompatible material that is inflexible at room temperature, and iii) a cutting device; and b) cutting the biocompatible material with the cutting device using the implant device pattern as a guide such that an implant device is generated, wherein the implant device comprises: i) a center plate that has: i) a length between about 4.0 and 40.0 millimeters, ii) a width between about 3.0 and 30.0 millimeters, and iii) a thickness of about 0.3 to about 5.0 millimeters (e.g. 0.6 millimeters), and ii) a plurality of protruding arms integral with the center plate, wherein the plurality of protruding arms project from the center plate in a planar manner. In certain embodiments, the cutting device is selected from a die cut steel rule press device, a laser cutting device, a Gerber cutting device, manual scissors, automatic scissors, a razor blade, or a knife.

### DESCRIPTION OF THE FIGURES

FIG. 1 shows an overhead view of an exemplary embodiment of the implant devices of the present invention.

FIG. 2A shows an overhead view of an exemplary embodiment of the implant devices of the present invention containing a plate hole 40, while FIG. 2B shows another exemplary embodiment of the implant devices of the present invention without a plate hole.

FIG. 3 shows one embodiment of an implant device of the present invention containing a plurality of protruding arms 30, where some of the protruding arms extend above a bone or bone defect and some extend below the bone or bone defect, such that the implant device is locked into place.

### DEFINITIONS

To facilitate an understanding of the invention, a number of terms are defined below.

As used herein, the terms “subject” and “patient” refer to any animal, such as a mammal like livestock, pets, and preferably a human. Specific examples of “subjects” and “patients” include, but are not limited to, individuals requiring surgery, and in particular, requiring endoscopic surgery to remove pituitary gland tumors.

As used herein, the terms “endoscopic surgery” and “endoscopic procedures,” and like terms, refer to what is generally known as laproscopic or endoscopic surgery, which generally involves indirect visualization of the operative field with a small camera (e.g. specialized fiberoptic telescopes measuring less than a half inch in diameter that are attached to high resolution television cameras). In preferred embodiments, endoscopic surgery to place the implantable devices of the present invention is performed through the nostril or nostrils of a patient.

As used herein, the phrase “planar manner” in reference to the protruding arms, indicates that the protruding arms extend from the center plate in the same plane as the center plate, or in a plane approximately parallel to the center plate.

As used herein, the phrase “inflexible at room temperature” refers to material that is rigid at room temperature such that it is generally resistant to bending.

### GENERAL DESCRIPTION OF THE INVENTION

The present invention provides implantable devices, and particularly implantable devices that may be positioned, for example, into the sphenoid sinus or sella turcica of a subject. In certain embodiments, the implantable devices comprise a center plate and a plurality of protruding arms extending from the center plate, both of which may be composed of bioresorbable material. In particular embodiments, the implantable devices are configured to help reconstruct the sellar floor after it has been damaged in order to prevent cerebrospinal fluid leaks.

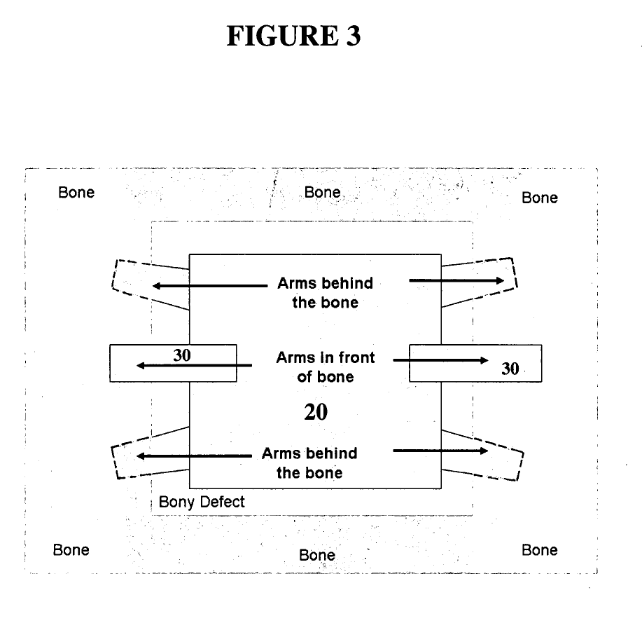

The implantable devices of the present invention have advantages over prior art implants. For example, the plurality of protruding arms allow the implant devices to be secured in place by locking to the edge of the skullbase, sella, sphenoid sinus, ethmoid sinus, frontal sinus or other bony edge. For example, the protruding arms may be positioned intradurally (inside the dura), between the dura and the edge of the bone, outside of the bony edge, or any combination (see, e.g., FIG. 3). The positioning of the plurality of protruding arms can be configured in relationship to the bone to allow the implant device to be secured in place.

### DETAILED DESCRIPTION OF THE INVENTION

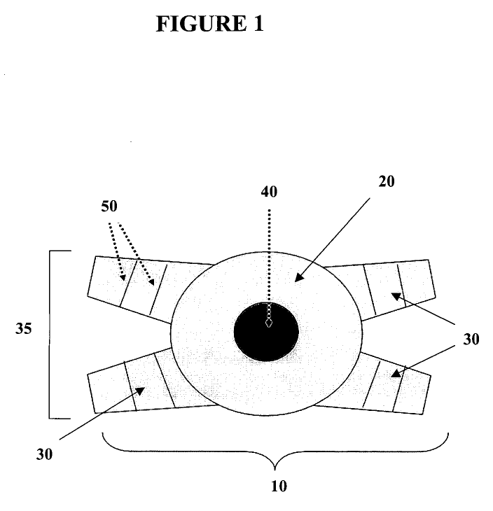

FIGS. 1-3 illustrate various preferred embodiments of the devices and related methods thereof. The present invention is not limited to these particular embodiments.

FIG. 1 shows an exemplary embodiment of an implantable device 10 according to the present invention. The implantable device 10 is shown with a center plate 20. The planer center plate 20 that is shown in FIG. 1 is circular. However, the center plate 20 may be any shape including, but not limited to, oval, square, rectangle, octagon, or any other shape. The center plate 20 is shown with a plate hole 40 in the center. The implantable devices may have any number of plates holes 40 (e.g. one, two, three, five, ten, etc.). Preferably, the plate holes 40 are configured such that the implantable device can be held with a surgical instrument inserted into the plate hole or plates holes. In certain embodiments, the plate hole or holes are configured to be grasped by forceps or similar instruments.

FIG. 1 also shows four protruding arms 30 attached to the center plate 20 such that they extend outward in a planner manner. While FIG. 1 shows four protruding arms 30, other numbers may be employed (e.g. two, three, five, or ten). In certain embodiments, the protruding arms 30 are present in pairs of protruding arms 35. Two such pairs of protruding arms 35 are shown in FIG. 1. The protruding arms 35 are shown in FIG. 1 with a number of grooves 50. Preferably, the grooves 50 are configured for trimming the protruding arms 35. For example, if a particular subject requires shorter arms in order to secure the implantable device into the sphenoid sinus or sella turcica, the protruding arms are cut along the appropriate grooves

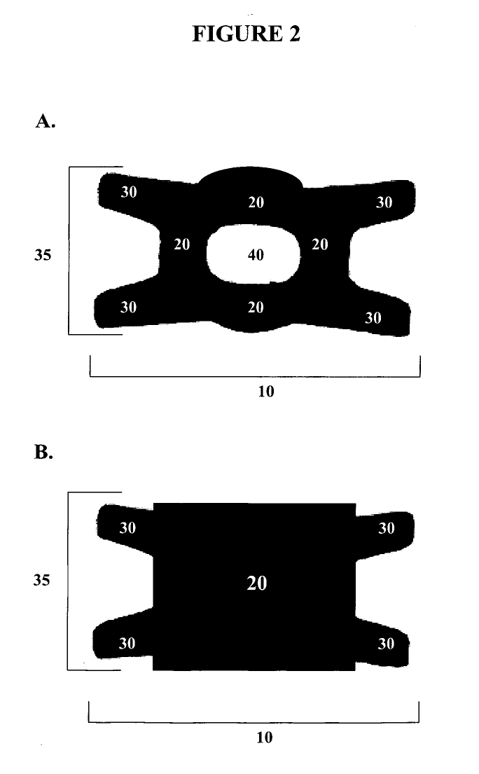

FIG. 2A shows another exemplary embodiment of an implantable device 10 according to the present invention. This figure shows an implantable device where the protruding arms 30 (and pairs of arms 35) are integral with the center plate 20. This figure also shows the center plate 20 with an oval shaped plate hole 40.

FIG. 2B shows an additional exemplary embodiment of an implantable device 10 according to the present invention. This figure shows an implantable device where the center plate 20 is in the shape of a square. The implantable device is also shown where the center plate does not contain a plate hole.

FIG. 3 shows one embodiment of an implant device of the present invention containing a plurality of protruding arms 30, where some of the protruding arms extend above a bone or bone defect and some extend below the bone or bone defect, such that the implant device is secured in place. The number and location of protruding arms 30 can be designed to accommodate a particular bone or boney defect.

In certain embodiments, the implantable devices of the present invention are configured for, and used in, reconstruction of the cranial base during or after transsphenoidal surgery. For example, the implantable devices may be used during such procedures to prevent the leakage of cerebrospinal fluid and/or to maintain anatomic integrity. In particular embodiments, the implantable devices are used for reconstruction of the floor of the sella turcica (e.g., following pituitary tumor removal). In some embodiments, the implantable devices are used in combination with autologous material, such as adipose tissue, muscle, or other tissue, to rebuild the floor of the sella turcica after it has been damaged.

In further embodiments, the implant devices of the present invention use other protruding members besides arms (e.g. other components that can be used to attach the implant devices to bone). In some embodiments, the implant devices contain a plurality of protruding fasteners, such as hooks, pins, screws, strings or similar fasteners.

All publications and patents mentioned in the above specification are herein incorporated by reference. Various modifications and variations of the described implant devices, compositions, methods, systems, and kits of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in art are intended to be within the scope of the following claims.

I claim:

1. An implant device comprising;
a) a center plate composed of a first bioresorbable material that is inflexible at room temperature, wherein said center plate has: i) a length between about 4.0 and 14.0 millimeters, ii) a width between about 3.0 and 13.0 millimeters, and iii) a thickness of about 0.3 to about 3.0 millimeters, and
b) a plurality of protruding arms composed of a second bioresorbable material that is inflexible at room temperature, wherein said plurality of protruding arms are attached to, or integral with, said center plate, and wherein said plurality of protruding arms project from said center plate in a planar manner.

2. The implant device of claim 1, wherein said first resorbable material comprises polylactide.

3. The implant device of claim 2, wherein said polylactide comprises poly D, L-lactide acid (PDLLA).

4. The implant device of claim 1, wherein said second resorbable material comprises polylactide.

5. The implant device of claim 4, wherein said polylactide comprises poly D, L-lactide acid (PDLLA).

6. The implant device of claim 1, wherein said center plate comprises at least one plate hole.

7. The implant device of claim 1, wherein said center plate does not have a plate hole.

8. The implant device of claim 1, wherein at least one of said plurality of protruding arms comprises grooves.

9. The implant device of claim 8, wherein said grooves are configured to facilitate cutting away a portion of said protruding arm.

10. The implant device of claim 1, wherein said implant device is configured to be placed into the sphenoid sinus of a subject.

11. The implant device of claim 1, wherein said implant device is configured to be placed into the sella turcica of a subject.

12. The implant device of claim 11, wherein said implant device is configured to aid in preventing cerebrospinal fluid leaks in said subject.

13. A method comprising;
a) providing;
i) a subject, and
ii) an implant device comprising a plurality of protruding arms, wherein said implant device is configured to be inserted into the skull base of said subject to aid in preventing cerebrospinal fluid leaks in said subject; and
b) inserting said implant device into said skull base of said subject.

14. The method of claim 13, wherein said skull base comprises the sphenoid sinus or sella turcica of said subject.

15. The method of claim 14, wherein said implant device is inserted into said sphenoid sinus of said subject.

16. The method of claim 13, wherein said inserting is via the nose of said subject.

17. The method of claim 13, wherein said implant device is inserted into said sella turcica of said subject.

18. A system comprising;
a) a sterilized implant device comprising;
i) a center plate composed of a first biocompatible material that is inflexible at room temperature, wherein said center plate has: i) a length between about 4.0 and 14.0 millimeters, ii) a width between about 3.0 and 13.0 millimeters, and iii) a thickness of about 0.3 to about 3.0 millimeters, and
ii) a plurality of protruding arms composed of a second biocompatible material that is inflexible at room temperature, wherein said plurality of protruding arms are attached to, or integral with, said center plate, wherein said plurality of protruding arms project from said center plate in a planar manner; and
b) a first sealed container, wherein said sterilized implant device is located inside said first sealed container, wherein said first sterilized implant device remains sterile while located inside said first sealed container.

19. The system of claim 18, further comprising c) a temperature indicator, wherein said temperature indicator visually indicates if said first sealed container has been exposed to a threshold temperature at any point.

20. The system of claim 19, further comprising an second sealed container, wherein said first sealed container is located inside of said second sealed container, and wherein said temperature indicator is affixed to said second sealed container.
